(19) United States
(12) Patent Application Publication
(10) Pub. No.: US 2004/0193264 A1
(43) Pub. Date: Sep. 30, 2004

(54) Compound AC IOL system

(76) Inventor: Richard Lindstrom, Wayzata, MN (US)

Correspondence Address: THOMAS R. FITZGERALD, ESQ., 16 E. MAIN STREET, SUTIE 210, ROCHESTER, NY, 14614-1803, US

(21) Appl. No.: 10/403,988

(22) Filed: Mar. 31, 2003

(51) Int. Cl. A61F 2/16

(52) U.S. Cl. 623/6.27; 623/6.36

(57) ABSTRACT

An IOL implant for correcting eyesight by placement into the anterior chamber of a patient wherein a relatively mild amount of correction is provided that his on the order of less than five dioplers. The implant can be designed for an astigmatism correction or the optic can be a multifocal lens, such as a bifocal lens. Haptics are provided allowing rotation of the implant during surgery to achieve the desired axis of correction for astigmatism.

### FIELD OF THE INVENTION

The present invention pertains to Intraocular Lenses, and more particularly to Intraocular Lenses that are placed in the anterior chamber of the eye.

### DESCRIPTION PRIOR ART

Implants of a intraocular lens (IOL) have been successfully used, most commonly to resolve cataracts by implantation of an IOL in the posterior chamber of the eye. This procedure, typically, involves removing the natural lens of the eye, and replacing it with an artificial lens. Other IOL implants have been used in the posterior chamber without removing the natural lens.

Recent advancements within intraocular lenses have been very substantial. IOL implants have evolved to include implants within the anterior chamber of the eye. Prior art IOL implants provided large amounts of correction, on the order of greater than+diopters of hyperopia (farsightedness) and more than −5 diopters of hyperopia (nearsightedness). However, the prior art remains deficient in the use of IOLs for relatively small amounts of correction in the range between −5 diopters and +5 diopters. Additionally, correction of astigmatism on the order of +5 diopters is also lacking within art of IOL implants.

Spherical refractive error is a reason for many IOC implants, a majority of these implants results in a certain degree of under correction or over correction of a sight for a patient. These patients suffering from residual refractive error, would benefit from additional correction that is not provided by the prior art.

Presbyopia remains in major source of eye impairment. Patients suffering from presbyopia would benefit from additional lens components, particularly, if the treatment was surgically reversible.

There is a needed within the art for intraocular lenses featuring ease-of-use and surgical reversibility.

### SUMMARY OF THE INVENTION

The present invention addresses the needs within the prior art by providing an intraocular lens (IOC) platform within the anterior chamber of the eye to correct refractive errors in psudophakic and phakic patients, as well as in presbyopic patients by the provision of an anterior chamber IOC that provides correction in a range of power between ±5D. This range of correction is not provided by prior art devices. The invention corrects (fine tunes) refractive errors in psudophakic patients. Psudophakic patients have already undergone cataract removal and corresponding implantation of a posterior chamber lens. A good majority of these patients have minor under (−) or over (+) corrections and could benefit from a combined IOL surgical treatment (implantation of lens in the AC of a patient already implanted with a posterior chamber lens) to correct their residual (spherical) refractive error. In addition to correcting residual spherical error, the invention is utilized to correct presbyopia by adding a bifocal/multifocal component to the lens. This approach provides a reversible treatment for presbyopia.

The invention provides anterior chamber IOL implant that is relatively simple to position during surgery and also allows for surgical reversibility. An anterior chamber lens absent of synecia is easier to implant and remove than a fibrosed posterior chamber lens in the capsular bag.

### BRIEF DESCRIPTION OF DRAWINGS

FIG. 1 is cross-sectional view of the human eye is viewed from the top;

FIG. 2 is view of anterior chamber intraocular lens implant as envisioned by the invention.

### DETAILED DESCRIPTION OF THE INVENTION

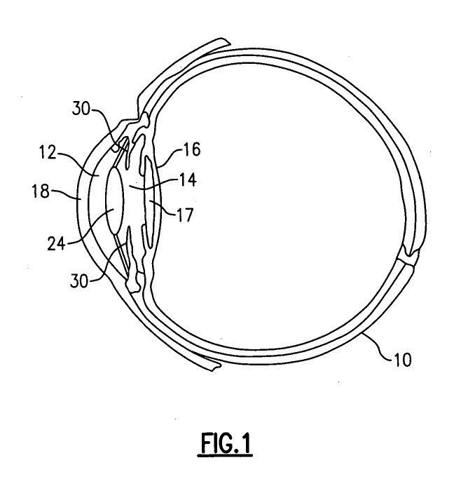

Referring to FIG. 1, which is a cross-sectional view of the human eye 10 as seen from above, an anterior chamber 12 and a posterior chamber 14 are separated by the iris 30. The posterior chamber 14 is a capsule 16 contains the natural crystalline lens 17 for the human eye 10. Light enters the eye by passing through the cornea 18 to the crystalline lens 17. A common procedure within the prior art is the removal of cataracts and the natural crystalline lens 17 and replacement implantation of an artificial lens referred to as an intraocular lens (IOL). Patients that have had their natural crystalline lens 17 replaced are referred to as psudophakic patients.

Psudophakic patients have an IOL in place of their natural crystalline lens 17 within the posterior chamber 14. A good majority of these psudophakic patients have minor under (−) or over (+) corrections and could benefit from a relatively small level to additional correction. The invention visions another IOL surgical implantation within the anterior chamber 12 for a patient that has a pre-existing IOL within the posterior chamber 14 to correct their residual (spherical) refractive error. The present invention of envisioned as placement of an IOL with correction in a range of powers from +5D to −5D to correct (fine tune) refractive errors in psudophakic patients. Besides correcting their residual spherical error, the technology could be utilized to correct presbyopia by adding a bifocal/multifocal component to the lens. The invention provides patients with an enhancement to the refractive results of a primary IOL implantation. Typically, second implants are done in the posterior chamber and not in the anterior chamber. The present invention provides for making second implants in the anterior chamber. The premise of the invention is to perform the implant to correct a small amount of error, in the range of −5D to +5D.

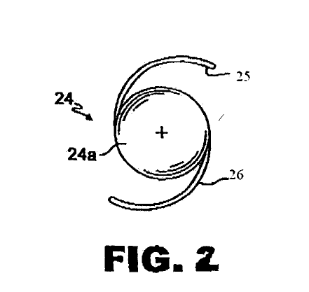

FIG. 2 is a diagram of the type of IOL 24 that is envisioned to be implanted within the anterior achamber 12. There are many different IOL designs used to facilitate different options for surgical placement of an IOL within an eye. The present invention concerns itself with fabrication and implanting of IOL 24 with substantially ovoid-shaped capsule 16 inside the anterior chamber 12 of the human eye 10. IOL 24 includes a central optic portion 24a and securing mechanisms for 25, 26 for securing the optic in a proper position within the anterior chamber 12. Securing mechanisms are preferably haptics, which are resilient structures extending radially outwardly from the periphery of the optic portion 24a. The IOL 24 of the invention as seen in FIG. 2 has two haptics, however, different numbers of haptics are envisioned to secure IOL 24 within the anterior chamber 12 of the human eye 10. The optic portion 24 can be centered using the soft springs (haptics) or by placing the optic portion connected to a soft sheet of material (not shown). For the reasons for the discussed below, haptics like those shown FIG. 2, are used within the preferred embodiment.

Another important feature of the invention allows simple and effective placement of a multifocal or bifocal optic as IOL 24. Preferably, multifocal or bifocal IOLs will be used in the anterior chamber with a posterior IOL already in place. It should be understood that the relatively small amount of correction is provided by the invention could also be used in the anterior chamber 12 without a pre-existing IOL device already in place.

Anterior chamber 12 IOL exist within the prior art, however, the anterior IOLs that are taught within the prior art, provide large amounts of correction. The present invention envisions implantation of an IOL with an optic portion 24a providing a small amount of correction within the anterior chamber 12 that is effective in correcting nearsightedness, farsightedness and presbyopia within a normal eye, or otherwise an eye for a phakic patient. The prior art has supplied very few teachings toward anterior chamber IOLs, and these few teachings have been for large correction IOL devices. The present invention provides an IOL implant that is useful for a large number of patients that can benefit from a small amount of correction that can be easily accomplished with little trauma using the IOL 24 of the invention within anterior chamber 12.

Preferably, the invention is a small Hydrogel, injectable, anterior chamber lens. Other materials, to silicon or acrylic, can be used in the construction of the IOL as envisioned by the present invention and the use of these other materials will be readily apparent to those skilled in the art. Prior art IOL devices that are placed within the anterior chamber 12 provide a greater degree of correction. The present invention is intended to be used primarily by patients that could benefit from correction but to a lesser extent. The prior art anterior chamber IOLs typically have a level of correction that is larger than the −5 to +5 range envisioned by the present invention.

About 1 to 2 percent of patients that receive IOL implants, such as bifocal implants, are not happy with the results. The reasons for these unhappy patients could be a difference in their night vision, resulting irritation from implant or a difference in depth perception. Even though these patients have improved vision, their individual preference of these patients is to have the implants removed. Therefore, an important feature for implants is the potential for surgical removability of the implant. In those cases where the implant is going to be surgically removed, it is desirable to remove the implant with as little trauma as possible.

The present invention provides desirable features for astigmatism and multifocal optics such as a bifocal optic implant.

The present invention is especially useful for the creation and implantation of a toric IOL implant that can be placed within the anterior chamber 12 to correct astigmatism. The lens is more accessible than implants placed within the posterior chamber. Placing the toric IOL within the anterior chamber 12 allows manipulation of the lens during implantation. It is relatively easy to rotate the lens until it rests upon the proper axis, which is an important feature in correcting astigmatism. For example, employing a 3 or 4 point fixation for the IOL 24 placed within the anterior chamber 12, the optic portion 24a that is envisioned by the present invention can be rotated by the surgeon until accurately placed. The optic can be rotated until the optic portion 24a rests on the desired axis to correct the astigmatism. Therefore, the present invention provides for IOL 24 that allows adjustment during the surgical procedure to achieve the desired amount of astigmatism correction. The surgeon can adjust the axis of the lens by simply adjusting and resetting the hooks on the haptics to achieve the desired correction for the astigmatism correction. This adjustment procedure can be performed during the primary procedure, or if necessary during a secondary procedure to precisely attenuate the amount of astigmatism correction.

The present invention expands the possibilities that are available for the phakic patients by providing a lens that can correct a middle ground range of problems. The present invention also expands the possibilities that available to psudophakic patients by providing a secondary procedure that can provide correction of residual refractive errors. These possibilities for phakic and a psudophakic patients is provided by a single lens that is placed within the anterior chamber, thus resulting in a simpler procedure.

Placement of the present invention is preferably accomplished with hooks, by placing a small stab like incisions in the cornea in the area where the cornea meets the limbus. Anterior lenses typically use haptics, which are essentially small hook like springs that attach the IOL during the implant processes. The haptics can be rotated and put into place resulting in a distinct advantage for the invention when placing toric lenses.

There are also issues related to IOL 24 implantation having a that bifocal lens for optic portion 24a into a patient. Occasionally, the patient is not happy with a bifocal lens. In that small number of cases where the patient desires to have the eye will removed the structure the present invention and placement in the anterior chamber 12 allows removal without causing significant trauma to the patient. Typically, a bifocal IOL made in accordance with the embodiments of the invention, can be removed with only topical anesthesia. Additionally, placement of the lens in the original procedure is accomplished using a lens that can be folded, such as Hydrogel, and inserted through a small incision in the cornea, thus providing a relatively simple and less traumatic procedure for placement of the IOL 24. Preferably, the lens of the present invention is Hydrogel, however numerous other lens materials will be. readily apparent to those skilled in the art. An advantage of the invention is the ease of use and surgical reversibility.

The embodiments most preferred to the inventor have been disclosed by the foregoing description, it will be readily apparent those who are skilled in the art that obvious variations of these embodiments are possible, therefore the scope of invention is to be measured by the appended claims.

1. A method of correcting eyesight by anterior chamber placement of an intraocular lends comprising the steps of:
providing the intraocular lens within an optic that provides of a correction of 4 dioplers or less, the optic having a plurality of placement mechanisms attached to the optic; and
placing the optic within the anterior chamber of a patient.

2. The method of claim 1 wherein the step of providing further comprises the correction being an astigmatism correction.

3. The method of claim 2 wherein the step of placing further comprises rotating the optic until a desired axis of correction is achieved.

4. The method of claim 1 wherein the step of providing further comprises a plurality of habics as the placement mechanisms.

5. The method of claim 1 wherein the step of providing further comprises the correction for the optic in a range of −5 dioplars for and +5 dioplars.

6. The method of claim 5, wherein the step of providing further comprises the optic being a multifocal lens.

7. The method of claim 6, wherein the step of providing further comprises the multifocal lens being a bifocal lens.

8. An IOL implant for placement within an anterior chamber comprising:
an intraocular lens having an optic that provides of a correction of 5 dioplers or less, the optic having a plurality of placement mechanisms attached to the optic; and
wherein, the optic is designed to be fastened by the placement mechanisms to the anterior chamber.

9. The implant of claim 8 wherein the correction is for astigmatism.

10. The implant of claim 9 wherein the placement mechanisms can be rotated within the anterior chamber until an axis of correction is at a desired point.

11. The implant of claim 8 wherein the placement mechanisms further comprises a plurality of habics.

12. The implant of claim 8 wherein the correction of the optic is in a range of −5 dioplars and +5 dioplars.

13. The implant of claim 12, wherein the optic further comprises the correction being a multifocal lens.

14. The implant of claim 13, wherein the optic further comprises the multifocal lens being a bifocal lens.
